(19) United States
(12) Patent Application Publication
(10) Pub. No.: US 2025/0170449 A1
(43) Pub. Date: May 29, 2025

(54) WEARABLE DEVICE AND METHOD OF MONITORING AND PROVIDING FEEDBACK OF AN EXERCISE ACTIVITY

(71) Applicant: KRJ ENTERPRISES LLC, Hueytown, AL (US)

(72) Inventor: Khiry R. Jennings, Hueytown, AL (US)

(21) Appl. No.: 18/964,229

(22) Filed: Nov. 29, 2024

Related U.S. Application Data
(60) Provisional application No. 63/604,126, filed on Nov. 29, 2023.

(51) Int. Cl. A63B 24/00; A63B 71/06

(52) U.S. Cl. CPC A63B 24/0006; A63B 71/0622; A63B 2024/0009; A63B 2071/0655; A63B 2220/40; A63B 2220/803; A63B 2220/836; A63B 2225/20; A63B 2225/50; A63B 2230/06; A63B 2230/40; A63B 2230/50; A63B 2230/65

(57) ABSTRACT

A wearable device and method of monitoring and providing feedback of an exercise activity is disclosed. The wearable device includes a wrist band, an ankle band, a strap, a bracelet, or other body part band. The wearable device includes a sensor for monitoring motion/position and pressure data of a user. The wearable device transmits the data to an electronic device. The electronic device monitors a number of characteristics related to the motion pattern of the exercise activity performed by the user and compares it with a predetermined pattern. If the motion pattern does not match with the predetermined pattern, then the electronic device generates a feedback with instructions related to correcting the execution of the exercise activity by the user. The electronic device provides step-by-step instructions of various workout routines for the user to follow, which helps to improve the quality and safety of the user's workout routines.

The present application claims the benefit of U.S. Provisional Application No. 63/604,126, filed Nov. 29, 2023; all of which is incorporated herein and referenced thereto.

### FIELD OF THE INVENTION

The present invention relates to wearable devices. More specifically, the present invention relates to a wearable device and method of monitoring and providing feedback of an exercise activity.

### BACKGROUND OF THE INVENTION

It is known that fitness enthusiasts or athletes would like to obtain feedback on their posture, movement and technique during exercise or playing sports. Typically, fitness enthusiasts or athletes use a trainer or coach to manually observe them performing the exercise or playing sports and provide feedback. In one example, the trainer or coach uses an image capturing device to record a video of the fitness enthusiast or athlete to analyse the posture, movement and technique and provides feedback.

With improvement in technology, fitness enthusiasts or athletes use wearable devices for measuring physical exertion parameters and use the data to obtain feedback. One such example is disclosed in a U.S. Pat. No. 10,065,074, entitled “Training systems with wearable sensors for providing users with feedback” (“the '074 Patent”). The '074 Patent discloses a training system based on mobile technology and kinematics of human motion characterizes, analyses, and supplies feedback to a user based on the user's movements. The training system includes a garment having a sensor control module connected to multiple sensor nodes via electrically-conductive fabric running along parts portions of the garment. The sensor module/nodes can communicate through the conductive fabric. The sensor nodes acquire motion and/or physiologic readings that are wirelessly transmitted to a mobile computing device that runs an application that analyses the data and provides visual (e.g., graphs, 3D avatar) and audio feedback (e.g., voice prompts). Vibration motors and LEDs/electroluminescent fabric in the garment also provide notifications and alerts. The triple layer of garment, conductive fabric, and sensor module/sensor node are sealed against contaminants, allowing the garment to be washable.

Another example is disclosed in a U.S. Pat. No. 10,799,760, entitled “System and method for identifying and interpreting repetitive motions” (“the '760 Patent”). The '760 Patent discloses a motion tracking system that monitors the motions performed by a user based on motion data received from one or more sensors. The motion tracking system may include a motion tracking device with one or more sensors, a smart device with one or more sensors and/or a server. As the user interacts with the motion tracking system or smart device the motion data generated by one or more sensors is processed by a software application. The software application generates interpreted data based on the motion data and contextual data such as the equipment being used by the user. Feedback is then provided to the user during and/or after the user has performed a motion or a set of motions. The feedback provided to the user may be visual, audio or tactile. The application may be used to monitor a routine in a sporting, fitness, industrial or medical environment, for example.

Yet another example is disclosed in a U.S. Pat. No. 10,417,932, entitled “Biometric data gathering” (“the '932 Patent”). The '932 Patent discloses a universal 6-DOF mems sensor combined with six degree of motion algorithms and human motion parameters permits individualized real time motion analysis of a user to enable accurate measurements. Data derived thereby is wirelessly sent for viewing to a Bluetooth® enabled smartphone or combination smartphone and eyeglass device, such as the Google Glass® headset. The sensor is worn on a wrist or ankle band or in combination with a chest mounted cardio heart rate monitor dependent on the biometric parameters measured. Typical physical exercise data gathered includes reps, sets, 10-100-yard dash times, vertical, horizontal and broad jump distances, a range of shuttle times, RAST, steps taken, distance traveled, velocity, acceleration, and calories burned. The heart rate monitor provides cardio assessment and the 6-DOF sensor measures a runner's pace and cadence data.

Although the above discussed devices are useful, they have few problems. For instance, most of the existing devices do not recognise and categorise different exercises leading to inaccurate feedback. Further, the existing devices are designed to track specific types of exercises e.g., running or cycling, and lack support for identifying a variety of exercises. As such, the existing devices are limited in their functionality. Furthermore, the existing devices do not tailor recommendations or feedback based on wearer's fitness levels and/or goals.

Therefore, there is a need in the art to provide an improved wearable device capable of monitoring and providing feedback on an exercise activity.

### SUMMARY

It is an object of the present subject matter to provide a wearable device capable of monitoring and providing feedback of an exercise activity and that avoids the drawbacks of known wearable devices.

It is another object of the present subject matter to provide a wearable device that monitors a number of characteristics related to the motion pattern of the exercise activity and provides suggestions to improve the quality and safety of user's workout routines.

In order to overcome one or more objects, the present subject matter presents a wearable device for monitoring and providing feedback of an exercise activity. The wearable device includes a wrist band, an ankle band, a strap, a bracelet, or other body part band. The wearable device includes a sensor for monitoring motion/position and pressure data of a user while performing the exercise activity. The wearable device transmits the data to an electronic device. The electronic device monitors a number of characteristics related to the motion pattern of the exercise activity performed by the user and compares it with a predetermined pattern. If the motion pattern does not match with the predetermined pattern, then the electronic device generates a feedback with instructions related to correcting the execution of the exercise activity by the user.

In one advantageous feature of the present subject matter, the wearable device/electronic device keeps track of the workout routines such as repetitions, sets and how much weight the user is using. This helps the user to review their performance and make adjustments, if any, to improve their workout routines.

In another advantageous feature of the present subject matter, the electronic device provides step-by-step instructions of various workout routines for the user to follow, which helps to improve the quality and safety of the user's workout routines.

The features and advantages of the subject matter here will become more apparent in light of the following detailed description of selected embodiments, as illustrated in the accompanying FIGURES. As will be realized, the subject matter disclosed is capable of modifications in various respects, all without departing from the scope of the subject matter. Accordingly, the drawings and the description are to be regarded as illustrative in nature.

### BRIEF DESCRIPTION OF THE DRAWINGS

A more complete understanding of the invention and its many advantages thereof will be readily appreciated as the same becomes better understood by reference to the following detailed description, when considered in connection with the accompanying drawings wherein:

FIG. 1 illustrates an environment in which a wearable device implements, in accordance with one embodiment of the present subject matter;

FIG. 2 illustrates a perspective view of the wearable device, in accordance with one embodiment of the present subject matter;

FIG. 3 illustrates a block diagram of the wearable device, in accordance with one embodiment of the present subject matter;

FIG. 4 illustrates a block diagram of an electronic device, in accordance with one embodiment of the present subject matter;

FIG. 5 illustrates a method of monitoring and providing feedback of an exercise activity, in accordance with one embodiment of the present subject matter;

FIG. 6 illustrates step-by-step instructions displayed on the electronic device for the user to perform the exercise activity, in accordance with one exemplary embodiment of the present subject matter; and

FIG. 7 illustrates an environment in which a wearable device implements, in accordance with another embodiment of the present subject matter.

### DETAILED DESCRIPTION

The following detailed description set forth below in connection with the appended drawings is intended as a description of exemplary embodiments in which the presently disclosed subject matter may be practiced. The term “exemplary” used throughout this description means “serving as an example, instance, or illustration,” and should not necessarily be construed as preferred or advantageous over other embodiments. The detailed description includes specific details for providing a thorough understanding of the presently disclosed wearable device. However, it will be apparent to those skilled in the art that the presently disclosed subject matter may be practiced without these specific details. In some instances, well-known structures and devices are shown in functional or conceptual diagram form in order to avoid obscuring the concepts of the presently disclosed wearable device.

In the present specification, an embodiment showing a singular component should not be considered limiting. Rather, the subject matter preferably encompasses other embodiments including a plurality of the same component, and vice-versa, unless explicitly stated otherwise herein. Moreover, the applicant does not intend for any term in the specification to be ascribed an uncommon or special meaning unless explicitly set forth as such. Further, the present subject matter encompasses present and future known equivalents to the known components referred to herein by way of illustration.

Although the present subject matter provides a description of a wearable device integrated, it is to be further understood that numerous changes may arise in the details of the embodiments of the wearable device. It is contemplated that all such changes and additional embodiments are within the spirit and true scope of this disclosure.

The following detailed description is merely exemplary in nature and is not intended to limit the described embodiments or the application and uses of the described embodiments. All of the implementations described below are exemplary implementations provided to enable persons skilled in the art to make or use the embodiments of the disclosure and are not intended to limit the scope of the disclosure.

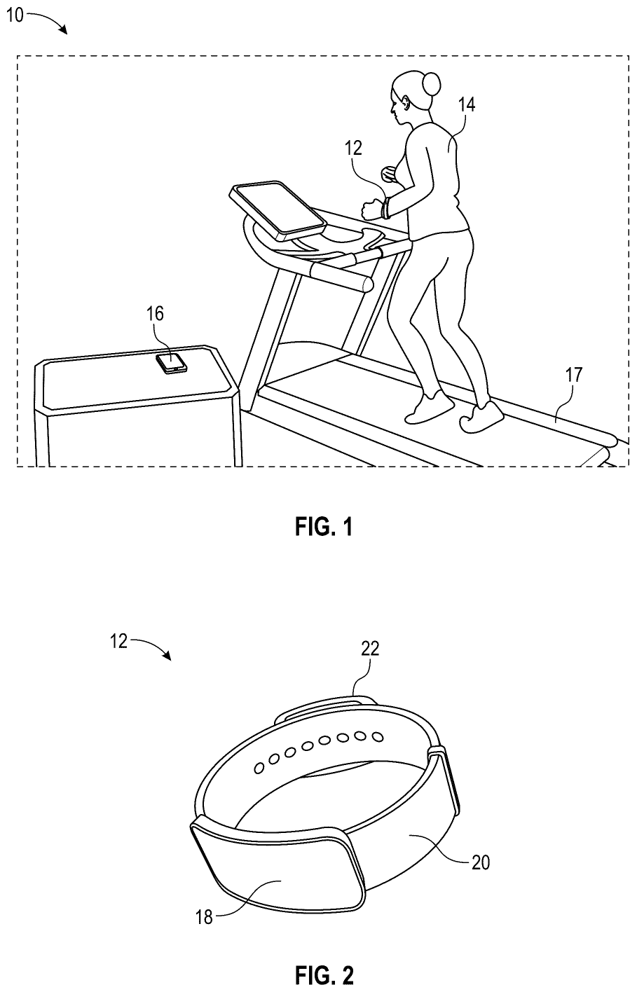

Various features and embodiments of a wearable device for monitoring and providing feedback of an exercise activity are explained in conjunction with the description of FIGUREs (FIGS. 1-7.

FIG. 1 shows an environment 10 in which a wearable device 12 implements, in accordance with one embodiment of the present subject matter. Wearable device 12 includes a wrist band, an ankle band, a strap, a bracelet, or other body part band. In one example, wearable device 12 comes in the form of a garment. Wearable device 12 is worn by a user 14 while performing an exercise activity. In accordance with the present subject matter, wearable device 12 monitors or senses biometric information from user 14 and transmits the information to an electronic device 16. Electronic device 16 includes a mobile phone, a laptop, a personal digital assistant or other suitable portable device. Exemplary biometric information that wearable device 12 monitors includes, but is not limited to, acceleration, movement, body position, heart rate, pulse rate, temperature, respiration, skin resistivity, orientation and rotation, etc. FIG. 1 shows user 14 running on exercise equipment 17 such as a treadmill wearing wearable device 12, in accordance with one exemplary embodiment of the present subject matter. However, a person skilled in the art understands user 14 can perform other exercise activities such as cycling, bicep curl, hammer curls, cycling, lifting weights, pain rehabilitation, yoga, etc.

FIG. 2 shows a perspective view of wearable device 12, in accordance with one embodiment of the present subject matter. As specified above, wearable device 12 comes in the form of a wrist band, an ankle band, a strap, a bracelet, or other body part band. Wearable device 12 includes a housing 18. Housing 18 is made of a metal, hard plastic or other suitable material. Wearable device 12 includes a strap 20 having a connecting mechanism 22. Strap 20 comes as a fabric strap, leather strap, metal strap, silicone strap, metal strap, etc. Connecting mechanism 22 helps to tighten strap 20 around the user's hand, ankle or any other body part. Connecting mechanism 22 includes a hook and loop mechanism, a buckle mechanism, a button mechanism, a magnetic mechanism, etc.

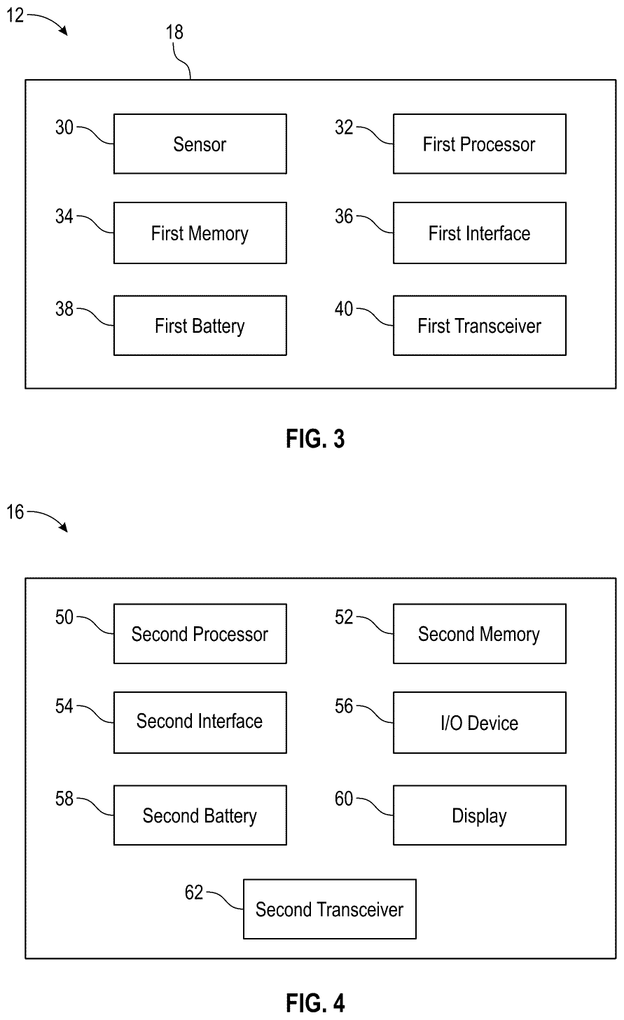

FIG. 3 shows a block diagram of wearable device 12, in accordance with one embodiment of the present subject matter. Housing 18 incorporates electronic components required to collect biometric information of user 14. Housing 18 includes a sensor 30. Sensor 30 includes, but not limited to, an accelerometer, a gyroscope, a heart rate monitor, a pressure sensor, a Micro Electronic Mechanical System (MEMS), a motion sensor, a location sensor, a Nano Electronic Mechanical System (NEMS) and an image capturing unit, etc. In one example, the presently disclosed sensor 30 is capable of measuring acceleration and movement of user 14. In another example, sensor 30 is capable of measuring heart rate during exercises. In another example, sensor 30 is capable of detecting specific movements and body positions of user 14 while performing the exercises. In another example, sensor 30 is capable of detecting location of user 14 while performing the exercises such as running or cycling.

Wearable device 12 includes a first processor 32 positioned in housing 18. First processor 32 includes one or more commonly known CPUs such as a microprocessor or a microcontroller. It should be understood that first processor 32 is responsible for implementing specific functions under the control of software including an operating system, and any appropriate applications software. Wearable device 12 includes a first memory 34 such as a volatile memory (e.g., RAM), non-volatile memory (e.g., disk memory, FLASH memory, EPROMs, etc.), unalterable memory, and/or other types of memory. In one implementation, first memory 34 is configured or designed to store data, program instructions. The program instructions control the operation of an operating system and/or one or more applications. Wearable device 12 includes a first interface 36. First interface 36 includes a wired interface and/or a wireless interface. In one implementation, first interface 36 includes functionality similar to at least a portion of functionality implemented by one or more computer system interfaces such as those described herein and/or generally known to one having ordinary skill in the art. In some examples, wearable device 12 includes a display (not shown) for providing first interface 36 to user 14 to operate wearable device 12.

Wearable device 12 includes a first battery 38. First battery 38 indicates a rechargeable battery such as a Lithium Ion (Li-ion) battery. First battery 38 is charged using a cable (not shown) via a charging port (not shown). Optionally, first battery 38 is charged wirelessly using inductive charging or charging pad (not shown) as known in the art. Further, wearable device 12 includes a first wireless communication module(s)/transceiver 40. First transceiver 40 is configured to communicate with external devices using one or more wireless interfaces/protocols such as, for example, 802.11 (Wi-Fi), 802.15 (including Bluetooth™), 802.15 (Wi-Max), 802.22, Cellular standards such as CDMA, CDMA2000, WCDMA, Radio Frequency (e.g., RFID), Infrared, Near Field Magnetics, etc.

FIG. 4 shows a block diagram of electronic device 16, in accordance with one embodiment of the present subject matter. Electronic device 16 includes a second processor 50. Second processor 50 includes one or more commonly known CPUs such as a microprocessor or a microcontroller. It should be understood that first processor 32 is responsible for implementing specific functions under the control of software including an operating system, and any appropriate applications software. Electronic device 16 includes a second memory 52 such as a volatile memory (e.g., RAM), non-volatile memory (e.g., disk memory, FLASH memory, EPROMs, etc.), unalterable memory, and/or other types of memory. In one implementation, second memory 52 is configured or designed to store data, program instructions. The program instructions control the operation of an operating system and/or one or more applications. Electronic device 16 includes a second interface 54. Second interface 54 includes a wired interface and/or a wireless interface. In one implementation, second interface 54 includes functionality similar to at least a portion of functionality implemented by one or more computer system interfaces such as those described herein and/or generally known to one having ordinary skill in the art. Electronic device 16 includes an Input/Output (I/O) device 56 such as a keyboard, buttons, etc.

Further, electronic device 16 includes a second battery 58. Second battery 58 indicates a rechargeable battery such as a Lithium Ion (Li-ion) battery. Second battery 58 is charged using a cable (not shown) via a charging port (not shown). Optionally, second battery 58 is charged wirelessly using inductive charging or charging pad as known in the art. Further, electronic device 16 includes a display 60 configured to display text or video. Further, electronic device 16 includes a second wireless communication module(s)/transceiver 62. Second transceiver 62 is configured to communicate with external devices using one or more wireless interfaces/protocols such as, for example, 802.11 (Wi-Fi), 802.15 (including Bluetooth™), 802.15 (Wi-Max), 802.22, Cellular standards such as CDMA, CDMA2000, WCDMA, Radio Frequency (e.g., RFID), Infrared, Near Field Magnetics, etc.

In one implementation, electronic device 16 includes an application or program. Launching the application connects to wearable device 12 and allows it to operate/access wearable device 12. In order to monitor and provide feedback of an exercise activity being performed by user 14, at first, user 14 connects electronic device 16 to wearable device 12. In one example, user 14 pairs electronic device 16 with wearable device 12 wirelessly using Bluetooth or Wi-Fi.

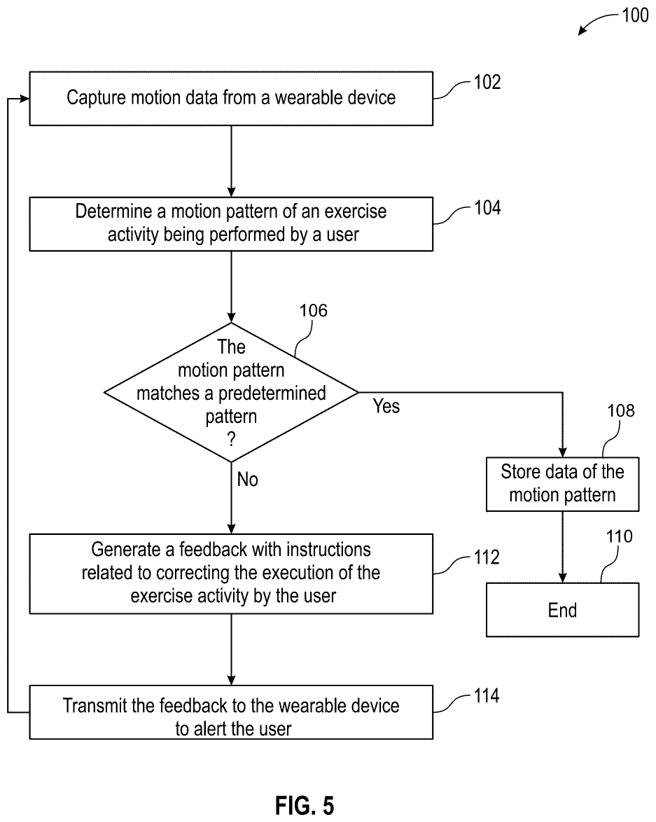

FIG. 5 illustrates a method 100 for monitoring and providing feedback of an exercise activity, in accordance with one exemplary embodiment of the present subject matter. The order in which method 100 is described should not be construed as a limitation, and any number of the described method blocks can be combined in any order to implement method 100 or alternate methods. Additionally, individual blocks may be deleted from method 100 without departing from the spirit and scope of the subject matter described herein. Furthermore, method 100 can be implemented in any suitable hardware, software, firmware, or combination thereof. However, for ease of explanation, in the embodiments described below, method 100 may be implemented using the above-described wearable device 12 and electronic device 16.

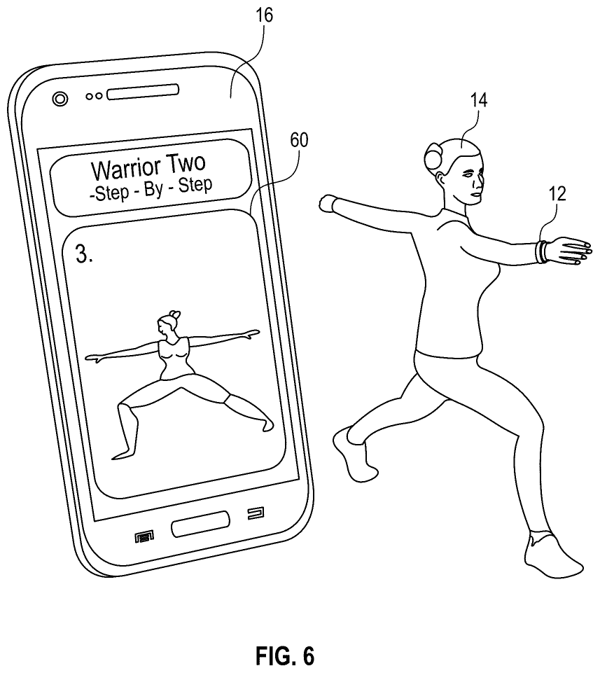

Here, user 14 activates wearable device 12 and performs the exercise activity. Optionally, wearable device 12 detects repeated motion or movements of user 14 and determines that user 14 has started the exercise activity. In one example, user 14 begins to perform the exercise activity based on a routine user 14 would like to perform. In another example, user 14 begins the exercise activity based on a routine suggested by electronic device 16. FIG. 6 shows electronic device 16 displaying step-by-step instructions with postures for performing the exercise activity such as yoga or Pilates, in accordance with one exemplary embodiment of the present subject matter. Consider an example where electronic device 16 suggests user 14 to perform five (5) sets of hammer curls using twenty (20) pound dumbbells and four (4) sets of ten (10) crunches with a rest period of 20 seconds between each exercise activity or set. Here, the suggestion provided by electronic device 16 is considered as a predetermined pattern. In one example, electronic device 16 suggests the routine for the exercise activity based on the user's body profile and age. Optionally, user 14 may predetermine the predetermined pattern which he/she wishes to follow.

As the user performs the exercise activity, sensor 30 monitors motion/position and pressure data of user 14, as shown at step 102. Further, first processor 32 instructs first transceiver 40 to transmit the data to electronic device 16. Electronic device 16 obtains the data in real-time and monitors a number of characteristics related to motion pattern of the exercise activity performed by user 14. Here, second processor 50 determines the type of exercise activity being performed by user 14, the quality of the form of user 14 as user 14 is performing the exercise activity and/or the number of counts or repetitions performed by user 14, as shown in step 104. At step 106, second processor 50 checks whether the motion pattern matches the predetermined pattern. If the motion pattern matches the predetermined pattern, method 100 moves to step 108. At step 108, electronic device 16 stores the motion pattern and displays the motion pattern on display 60 after user 14 completes a session of the exercise activity. At step 110, method 100 ends.

If the motion pattern does not match with the predetermined pattern at step 106, then method 100 moves to step 112. At step 112, electronic device 16 generates a feedback with instructions related to correcting the execution of the exercise activity by user 14. For example, consider user 14 is performing four (4) sets of hammer curls using twenty (20) pound dumbbells, then electronic device 16 generates the feedback that user 14 is doing only four (4) sets of hammer curls instead of recommended five (5) sets of hammer curls. In another example, consider user 14 is not taking rest or taking rest less than 20 seconds between each exercise activity or set, then electronic device 16 generates the feedback to take 20 seconds rest between the exercise activity or set. In one example, electronic device 16 transmits the feedback and displays on display 60 for user 14 to follow, as shown in step 114. Optionally, electronic device 16 transmits a haptic feedback to wearable device 12 to provide physical stimuli such as vibration to simulate tactile experience for user 14 to correct the exercise activity being performed by user 14. In one example, electronic device 16 provides the feedback to user 14 in real-time as user 14 performs the exercise activity causing wearable device 12 to vibrate in order to notify user 14 of bad/irregular form as user 14 is performing a curl. After suggesting the feedback, method 100 moves back to step 102 whereby wearable device 12 captures the motion and pressure data from wearable device 12. In one example, the feedback is displayed in the form of charts or tables on display 60 showcasing the performance of user 14 through the exercise activity or fitness routine.

Although the above method 100 is explained considering that wearable device 12 sends the motion data to electronic device 16 and electronic device 16 processes the motion data and provides the feedback, it is possible to configure wearable device 12 such that first processer 32 processes the motion and pressure data captured by sensor 30 and provides the feedback via the display (not shown) or causes the wearable device 12 to provide physical stimuli without departing from the scope of the present subject matter. In other words, wearable device 12 operates as a standalone device without the need for electronic device 16.

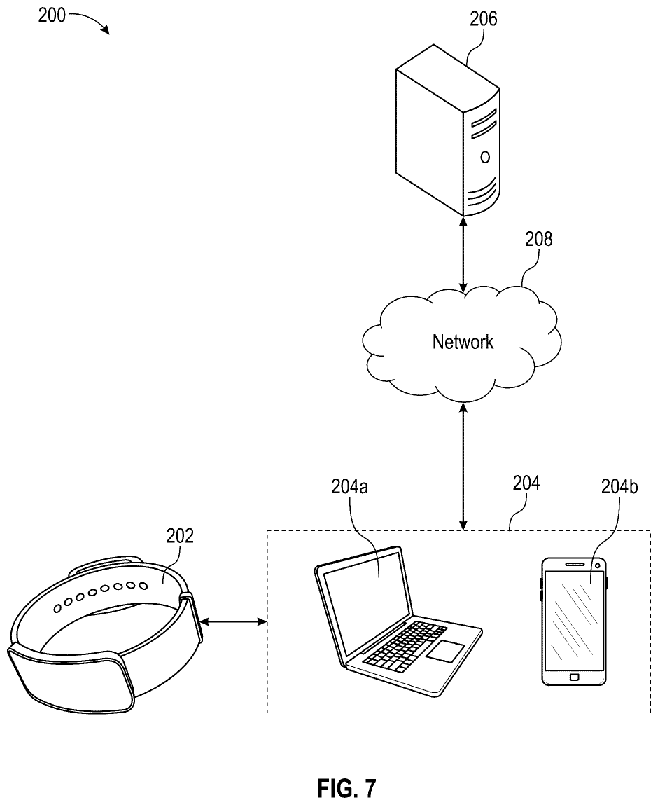

Now referring to FIG. 7, an environment 200 in which a wearable device 202 implements is shown, in accordance with another embodiment of the present subject matter. Wearable device 202 includes and operates similar to wearable device 12 explained above. Wearable device 202 monitors or senses biometric information from a user (not shown) and transmits the information to an electronic device 204 (similar to electronic device 16). Electronic device 204 includes a first electronic device 204a such as a laptop and a second electronic device 204b such as a mobile phone, collectively referred to as electronic devices 204 or simply electronic device 204. Electronic device 204 communicatively connects to a server 206 via a network 208. Here, network 208 may include a wireless network, a wired network or a combination thereof. Network 208 can be implemented as one of the different types of networks, such as intranet, local area network (LAN), wide area network (WAN), the internet, and the like. Network 208 may either be a dedicated network or a shared network. The shared network represents an association of the different types of networks that use a variety of protocols, for example, Hypertext Transfer Protocol (HTTP), Transmission Control Protocol/Internet Protocol (TCP/IP), Wireless Application Protocol (WAP), and the like, to communicate with one another. Further network 208 may include a variety of network devices, including routers, bridges, servers, computing devices, storage devices, and the like.

Server 206 is operated by a fitness band manufacturer or athlete performance service provider, etc. In the present embodiment, electronic device 204 receives the motion data and transmits to server 206. Server 206 processes the data and records the motion data. Further, server 206 analyses the motion data and sends feedback to be displayed on electronic device 204, as explained above.

The presently disclosed wearable device provides several advantages over the prior art. The wearable device can be worn by the user on his/her wrist, arms, elbows, knees, calves, shins, ankles, etc. The wearable device allows for monitoring movements and stresses on the body during a workout routine. The wearable device transmits the real-time data to the electronic device, which processes and provides suggestions/feedback in real-time for the user to correct the posture, stance, etc. when an inappropriate motion or pressure is detected. Further, the wearable device provides step-by-step instructions of various workout routines for the user to follow, which helps to improve the quality and safety of the user's workout routines.

A person skilled in the art appreciates that the wearable device can come in a variety of shapes and sizes depending on the need and comfort of the user. Further, many changes in the design and placement of components may take place without deviating from the scope of the presently disclosed wearable device.

In the above description, numerous specific details are set forth such as examples of some embodiments, specific components, devices, methods, in order to provide a thorough understanding of embodiments of the present subject matter. It will be apparent to a person of ordinary skill in the art that these specific details need not be employed, and should not be construed to limit the scope of the subject matter.

In the development of any actual implementation, numerous implementation-specific decisions must be made to achieve the developer's specific goals, such as compliance with system-related and business-related constraints. Such a development effort might be complex and time-consuming, but may nevertheless be a routine undertaking of design, fabrication, and manufacture for those of ordinary skill. Hence as various changes could be made in the above constructions without departing from the scope of the invention, it is intended that all matter contained in the above description or shown in the accompanying drawings shall be interpreted as illustrative and not in a limiting sense.

The foregoing description of embodiments is provided to enable any person skilled in the art to make and use the invention. Various modifications to these embodiments will be readily apparent to those skilled in the art, and the novel principles and invention disclosed herein may be applied to other embodiments without the use of the innovative faculty. It is contemplated that additional embodiments are within the spirit and true scope of the disclosed subject matter.

What is claimed is:

1. A method of monitoring and providing feedback on an exercise activity, said method comprising the steps of:
acquiring, by a processor, biometric information of a user performing an exercise activity using a sensor;
determining, by said processor, a motion pattern of the exercise activity based on the biometric information acquired;
comparing, by said processor, the motion pattern with a predetermined pattern; and
providing, by said processor, a haptic feedback to present a physical stimuli in order to simulate a tactile experience for said user to correct the exercise activity being performed by said user based on the comparison.

2. The method of claim 1, further comprising:
identifying deviations in the motion pattern from the predetermined pattern; and
providing real-time instructions for correcting the exercise activity.

3. The method of claim 2, wherein the real-time instructions comprise visual instructions, and auditory instructions, or a combination thereof.

4. The method of claim 1, further comprising analyzing, by said processor, the biometric information for recommending an exercise routine based on an age and profile of said user.

5. The method of claim 1, wherein the biometric information comprises acceleration data, movement data, body position data, heart rate, pulse rate, temperature, respiration, skin resistivity, or a combination thereof.

6. The method of claim 1, wherein the predetermined pattern comprises recommended exercise form, specific number of exercise sets; specific number of repetitions; and rest period duration between sets.

7. The method of claim 1, further comprising detecting, by said processor, repeated motion or movement corresponding to the exercise activity performed by said user prior to the step of acquiring the biometric information.

8. A wearable device for monitoring and providing feedback on an exercise activity, said wearable device comprising:
a processor; and
a memory coupled to said processor, wherein said processor is configured to execute program instructions stored in said memory, to:
acquire biometric information of a user performing an exercise activity using a sensor;
determine a motion pattern of the exercise activity based on the biometric information acquired;
compare the motion pattern with a predetermined pattern; and
provide a haptic feedback to present a physical stimuli in order to simulate a tactile experience for said user to correct the exercise activity being performed by said user based on the comparison.

9. The wearable device of claim 8, wherein said processor executes the program instructions to detect repeated motion or movement corresponding to the exercise activity performed by said user prior to the step of acquiring the biometric information.

10. The wearable device of claim 8, wherein said processor executes the program instructions to identify deviations in the motion pattern from the predetermined pattern, and provide real-time instructions for correcting the exercise activity.

11. The wearable device of claim 8, wherein said processor executes the program instructions analyze the biometric information to recommend an exercise routine based on an age and profile of said user.

12. The wearable device of claim 10, wherein the real-time instructions comprise visual instructions, and auditory instructions, or a combination thereof.

13. The wearable device of claim 12, wherein the visual instructions are displayed on a display of said wearable device.

14. The wearable device of claim 8, wherein the biometric information comprises acceleration data, movement data, body position data, heart rate, pulse rate, temperature, respiration, skin resistivity, or a combination thereof.

15. The wearable device of claim 8, wherein the predetermined pattern comprises recommended exercise form, specific number of exercise sets; specific number of repetitions; and rest period duration between sets.

16. The wearable device of claim 8, wherein said processor executes the program instructions to transmit the biometric information to a server for analyzing the biometric information.

17. The wearable device of claim 8, wherein said processor executes the program instructions to transmit the biometric information to an electronic device for analyzing the biometric information, and wherein said electronic device comprises one of a mobile phone; a laptop; a personal digital assistant; and a tablet computer.

18. The wearable device of claim 8, wherein said sensor comprises one of an accelerometer, a gyroscope, a heart rate monitor, a pressure sensor, a Micro Electronic Mechanical System (MEMS), a motion sensor, a location sensor, a Nano Electronic Mechanical System (NEMS) and an image capturing unit.

19. The wearable device of claim 8, wherein said wearable device comprises one of a wrist band, an ankle band, a strap, and a bracelet.

20. A non-transitory, computer-readable medium storing instructions that, when executed by a computer system for monitoring and providing feedback on an exercise activity, configure the computer system for:
acquiring biometric information of a user performing an exercise activity using a sensor;
determining a motion pattern of the exercise activity based on the biometric information acquired;
comparing the motion pattern with a predetermined pattern; and
providing a haptic feedback to present a physical stimuli in order to simulate a tactile experience for said user to correct the exercise activity being performed by said user based on the comparison.
